(19) United States
(12) Patent Application Publication
(10) Pub. No.: US 2019/0376016 A1
(43) Pub. Date: Dec. 12, 2019

(54) PERSONALIZED CELLULAR BIOMANUFACTURING WITH A CLOSED, MINIATURE CELL CULTURE SYSTEM

(71) Applicant: NUtech Ventures, Lincoln, NE (US)

(72) Inventor: Yuguo Lei, Lincoln, NE (US)

(21) Appl. No.: 16/462,753

(22) PCT Filed: Nov. 22, 2017
(86) PCT No.: PCT/US17/63036

§ 371 Date: May 21, 2019

Related U.S. Application Data
(60) Provisional application No. 62/425,141, filed on Nov. 22, 2016.

(51) Int. Cl. C12M 1/12; C12N 5/074; C12N 5/0735; G01N 33/50

(52) U.S. Cl. CPC C12M 25/14; C12N 5/0696; C12N 2533/30; G01N 33/50; C12N 2513/00; C12N 5/0606

(57) ABSTRACT

Closed, miniature devices and methods of using the devices for culturing cells are disclosed. Particularly, a device, and methods of using the device for manufacturing, expanding, differentiating and/or reprogramming cells for personalized medicine, such to allow for conducting medical procedures at the point-of-care, are provided.

### CROSS-REFERENCE TO RELATED APPLICATION

This application claims priority to U.S. Provisional Patent Application No. 62/425,141, filed Nov. 22, 2016, which is incorporated by reference herein in its entirety.

### BACKGROUND OF THE DISCLOSURE

The present disclosure relates generally to personalized culturing, reprogramming, expanding, differentiating, and/or downstream processing of cells, such as primary human cells, primary human tumor cells, human pluripotent stem cells (hPSCs) (including human induced pluripotent stem cells (hESCs) and human embryonic stem cells (iPSCs)) and their derivatives (i.e. cells differentiated from hPSCs) in a closed, miniature cell culture system. More particularly, the present disclosure relates to a closed device for manufacturing, expanding, differentiating and/or reprogramming cells for personalized medicine, such to allow medical procedures at the point-of-care (i.e., at the time and place of patient care).

Autologous cells refer to cells from the patient, and thus are attractive for use in cellular therapies as they induce minimal or no immune rejection after transplanting to the patient. Autologous cells include primary cells isolated from the patient, such as T cells, chondrocytes, and mesenchymal stem cells. These cells can be used to treat many human diseases that cannot be treated, or their progression cannot be altered by current treatments.

Autologous cells also include patient specific human induced pluripotent stem cells (iPSCs). By delivering a few reprogramming factors into the cells, adult cells from the patient (e.g., fibroblasts) can be reprogrammed into iPSCs within about one month. iPSCs can be cultured for long durations and expanded into large numbers under completely defined conditions. They can be further differentiated into presumably all the cell types of the human body.

Autologous cells also include primary tumor cells from the patient, such as glioblastoma cells. These cells can be used to screen drugs that can specifically and efficiently kill the patient's tumor cells.

Autologous cell-based personalized medicine, however, cannot benefit the large patient population until they become affordable. The expense to biomanufacture personalized cells with current technologies and bioprocess are extremely high. For instance, to make patient specific iPSC-based autologous cells with the current bioprocessing, patient cells are collected and cultured for a few days; then, reprogramming factors are delivered to these cells to reprogram them into iPSCs (which takes approximately one month). Next, high quality iPSC clones are selected, expanded and characterized for their pluripotency and genome integrity with a variety of assays (which takes approximately one to two months); then, iPSCs are expanded and differentiated into the desired cells. Finally, the produced cells are purified, characterized for their identities, purity, and potency, and formulated for transplantation. The whole bioprocessing takes a few months and is mainly done using 2D, open culture systems (e.g., 2D cell culture flasks) through manual operations—a processing which leads to low reproducibility, high risk of contamination, and requirement for highly skilled technicians. In addition, 2D culture systems have low yield. For instance, only ˜2×10⁵ cells can be produced per cm² surface area, meaning that it would require ˜85 six-well plates to produce the cells (−1×10⁹ cells) sufficient for one patient. Maintaining these plates requires large incubators and cGMP facility space, labor, and reagents.

If large numbers of patients need iPSC-based personalized cell therapies, the cell production can only be done in large cell biomanufacturing centers (i.e. centralized cellular biomanufacturing). Patient cells are sent to the center, and the produced cells are sent back to the point-of-care for transplantation. This centralized biomanufacturing has additional disadvantages, including: (i) cross-contamination and (ii) high costs and risks associated with the transportation, logistics, tracking, and recording. In summary, the cost for biomanufacturing personalized iPSCs and their derivatives with current technologies is not affordable for the majority of patients.

One method to significantly reduce the biomanufacturing cost is to automate the bioprocessing in individualized, closed, computer controlled miniature cell culture devices to biomanufacture the cells at the point-of-care (i.e. cGMP-in-a-box production). Using closed culture devices avoids contamination risk and eliminates the requirement for cGMP processing. Automation of all key operations avoids output variations and reduces the need for highly skilled operators. Biomanufacturing at the point-of-care reduces the cost and risk related to the logistics and transportation. Miniaturizing the culture system makes it possible to simultaneously biomanufacture cells for large numbers of patients at the point-of-care (i.e. high throughput biomanufacturing).

Based on the foregoing, there is a need in the art for a closed, miniature device for manufacturing, expanding, differentiating and reprogramming cells, particularly on a scale such that can be used at the point-of-care for personalized medicine. It would further be advantageous if the closed device could be made to be disposable to limit cross-contamination.

### BRIEF DESCRIPTION OF THE DISCLOSURE

The present disclosure is generally directed to culturing, reprogramming, expanding, differentiating and downstream processing cells in a closed culture system. More particularly, the present disclosure is directed to a closed culturing system and device including a closed housing that can be used for manufacture, expansion, differentiation of cells, and then further, for concentration, purification and transportation of the cultured cells.

In one aspect, the present disclosure is directed to a device for culturing cells for personalized medicine, the device comprising: a closed housing comprising a three-dimensional (3D) hydrogel scaffold; an inlet for introducing a cell culture medium into the housing; and an outlet for exhausting cell culture medium from the housing.

In another aspect, the present disclosure is directed to a method of expanding cells using the device, the method comprising: suspending a cell solution including cells in the 3D hydrogel scaffold of the closed housing; introducing a cell culture medium into the closed housing from the inlet; and culturing the cells.

In yet another aspect, the present disclosure is directed to a method of differentiating cells using the device, the method comprising: suspending a cell solution including cells in the 3D hydrogel scaffold of the closed housing; introducing a cell differentiation medium into the closed housing from the inlet; and culturing the cells.

In another aspect, the present disclosure is directed to a method of reprogramming cells using the device, the method comprising: suspending a cell solution including adult cells in the 3D hydrogel scaffold of the closed housing; introducing a cell culture medium into the closed housing from the inlet; and reprogramming the cells.

In accordance with the present disclosure, methods have been discovered that surprisingly allow for culturing, manufacturing, expanding, differentiating and reprogramming cells in a closed, miniature culture system. The methods and devices of the present disclosure will have significant impact on personalized medicine as they allow for sufficient, high quality and affordable cells that can be used at the point-of-care. Further, the devices and methods provide an advantageous impact on the biopharmaceutical industry by providing more affordable methods for manufacturing, expanding, differentiating and reprogramming cells in a manner that limits contamination and cross-contamination.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A-1C depict a closed, miniature cell culture device for personalized cellular biomanufacturing as described in the present disclosure. FIG. 1A depicts a schematic illustration of the device. FIG. 1B is a picture of the cell culture device with the inlet and outlet identified. FIG. 1C is a picture of the cell mass in hydrogel fibers within the cell culture device.

FIGS. 2A-2C depict personalized iPSC expansion and differentiation into neural stem cells (NSCs) in a closed, miniature cell culture device. FIG. 2A illustrates the methods of the bioprocessing as described in the present disclosure. FIG. 2B depicts the miniature cell culture device 210 including a pump 212 for medium perfusion, an oxygen-permeable plastic bag 214 for stocking medium and a closed 15-ml conical tube 216. Further, fibrous hydrogel fibers with cells are shown suspended in the tube. FIG. 2C depicts mixing single iPSCs with a 10% PNIPAAm-PEG solution at 4° C. on day 0 and injected into room temperature cell culture medium in a 15-mL conical tube to instantly form hydrogel fibers with cells; culturing the cells in E8 medium for 5 days; culturing the cells for an additional 7 days in neural induction medium in the conical tube to differentiate the cells (medium was continuously perfused); liquefying the hydrogel scaffolds by placing the cell culture tube on ice for 5 minutes; pelleting the cell spheroids by spinning the tube at 100 g for 3 minutes (medium was removed); purifying the cells; adding magnetic beads coated with anti-SSEA4 antibodies into the tube to pull down the undifferentiated SSEA4+ iPSCs with a magnetic cell separator; transferring the purified cells in the supernatant into a new, closed tube, and transporting the closed tube to the surgical room; and transplanting the NSCs to rat brain with a stereotactic injector. Specifically, as shown in the purifying step, cell spheroids were incubated in Accutase at 37 ° C. for 10 minutes. The reagents were removed from the tube and new reagents were added to the tube with a sterile syringe through the septum cap.

FIGS. 3A-3E depict cells in the miniature bioprocessing method of the present disclosure. FIG. 3A are phase images of the hydrogel fibers and cells on day 0, 5 and 12 of the bioprocessing. FIG. 3B depict Live/dead staining of cells on day 12. FIG. 3C show that ˜97% of the purified cell products expressed NSC markers, PAX6 and Nestin. FIG. 3D show that cells pulled down by the magnetic anti-SSEA4 beads were positive for Oct4 and Nanog. FIG. 3E show that HuNu+ (human nuclear antigen) NSCs survived well in the rat brain 7 days post-transplantation.

FIGS. 4A-4E depict culturing cells in alginate hollow fibers as described in the present disclosure. FIG. 4A is a schematic showing a hyaluronic acid (HA) solution containing single cells 320 and alginate solution 322 pumped into the central 324 and side channels 326 of a home-made micro-extruder, respectively, to form a coaxial core-shell flow that is extruded into a CaCl₂ buffer 328 (100 mM), which instantly crosslinks the alginates to form hydrogel shells to make hollow fibers. Subsequently, CaCl₂ buffer was replaced by cell culture medium and cells were suspended and grown in the core microspace of the hollow fibers. FIG. 4B shows that, within the first 24 hours, the single cells associated to form small clusters (i.e., initial clustering phase). Subsequently these small clusters expanded as spheroids (FIG. 4C) that eventually merge to form cylindrical cell masses (FIG. 4D) (i.e., cell growth phase). FIG. 4E depict a cylindrical cell mass in one hollow fiber on day 9.

FIGS. 5A & 5B depict personalized iPSC expansion and differentiation into NSCs in a closed, miniature cell culture device using alginate hydrogel hollow fibers as described in the present disclosure. FIG. 5A depicts a schematic illustration of the bioprocess. As shown in FIG. 5B, iPSCs and hydrogel fibers were extruded into a closed 15-ml tube; iPSCs in the hollow fibers were expanded for 5 days in the expansion medium with automated medium perfusion. iPSCs were then differentiated into NSCs in the differentiation medium for 7 days. Fibers were dissolved by adding 0.5 mM EDTA, and cell spheroids were harvested by gravity. Spheroids were then dissociated into single cells with Accutase. Undifferentiated iPSCs were depleted with magnetic anti-SSEA-4 beads. The cell products were transferred to a new tube and concentrated by centrifugation. Cells were transported to the surgery room and transplanted.

FIGS. 6A-6J depict iPSC expansion and differentiation into NSCs in a miniature bioprocess using alginate hydrogel hollow fibers as described in the present disclosure. FIG. 6A are phase images of cells growing in hydrogel fibers on day 0 (single iPSCs), day 5 (iPSC spheroids) and day 5+7 (NSC aggregates). On day 5+7, 400-fold of expansion (FIG. 6B), yield of 4.1×10⁸ cells/ml (FIG. 6C), >95% cell viability were achieved (FIG. 6D). 98% of cells were SSEA negative (FIG. 6E); and very few dead cells (via live/dead cell staining) were detected (FIG. 6F). FIGS. 6G & 6H show that >99% of the cells pulled down by the anti-SSEA4 antibody-coated magnetic beads were Nanog+/Oct4+ undifferentiated iPSCs. FIG. 6I shows that >99% of the purified cell products were PAX6+ /Nestin+ NSCs. FIG. 6J shows that purified NSCs survived well in mouse brain 7 days after transplantation. HuNu: human nuclear antigen.

FIG. 7 depicts iPSC colonies formed in the 3D hydrogels used in the devices of the present disclosure after 3 weeks of reprogramming

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, devices and methods have been discovered that surprisingly allow for the culturing, reprogramming, expanding, differentiating and downstream processing of cells in a closed, miniature system such to allow for limited contamination, lower costs, high cell yield and purity, and ease of providing personalized medicine. Particularly, the present disclosure provides a closed, miniature device and methods of using the device for manufacturing, expanding, differentiating and reprogramming cells in a closed, miniature system using 3D hydrogel scaffolds.

### Device for Culturing/Manufacturing/Expanding/Differentiating/Reprogramming Cells

Advantageously, the device of the present disclosure allows for biomanufacturing sufficient and affordable personalized cells at the point of care. Further, the device provides high cell yields and purity while limiting contamination. Generally, the device includes a closed, miniature housing including hydrogel scaffolds with cells; an inlet with filter for flowing cell culture medium into the housing; and an outlet with filter for flowing out of the housing the exhausted medium. As used herein, “miniature” refers to the device including a housing having a capacity of less than 10 L, including from about 1 ml to less than 10 L, including from about 1 ml to about 1000 ml in capacity.

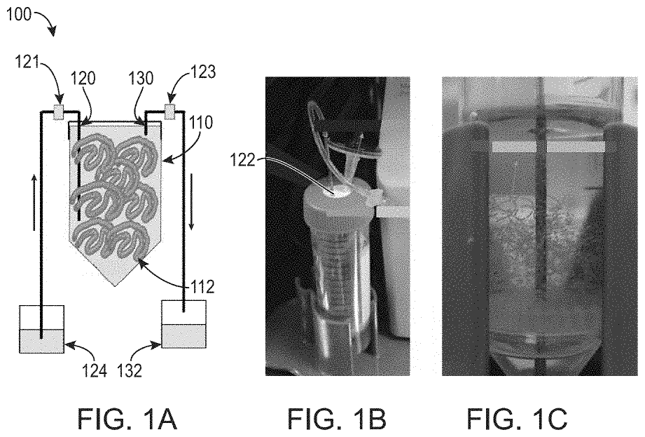

More particularly, as shown in FIG. 1A, the device 100 includes a closed housing 110; an inlet 120 and an outlet 130. As used herein, “closed” as referred to in “closed device”, “closed system”, and/or “closed housing” refers to the device, system, and/or housing that is sealed such that the exchange of matter with its surroundings can only be done through the inlet and outlet with filters, 121, 123. The filters 121, 123 can prevent the virus and bacteria in the environment from entering the cell culture device. More particularly, the closed device, system, and/or housing suitably prevents at least 70% of surrounding matter from entry into the device, system, and/or housing; more suitably, at least 75%; even more suitably, at least 80%; even more suitably, at least 90%; even more suitably, at least 95%, including 96%, 97%, 98%, 99%, and even 100% of surrounding matter from entry into the device, system and/or housing.

The closed housing 110 as shown in FIG. 1A is a closed 50-ml conical tube; however, it should be understood by one skilled in the art that any closed culture system known in the art, for example larger conical tubes or small volume plastic bags. Typically, when a conical tube is used, the tube is sized to a capacity of from 1 ml to about 10 L, including from about 1 ml to about 1 L, and including from about 5 ml to about 50 ml. When plastic bags are used, the bags have a capacity of from about 1 ml to about 10 L and including from about 1 ml to about 1 L.

The closed housing 110 includes a three-dimensional (3D) hydrogel scaffold 112. The 3D hydrogel scaffold is prepared by extruding the hydrogel precursor solution with cells through the septum cap 122 (FIG. 1B) of the cell culture device into a buffer containing crosslinking reagents in the cell culture device that can quickly crosslink the hydrogel precursor solution into hydrogels.

Typically, the 3D hydrogel scaffold 112 is prepared using any polymers as known in the hydrogel art for culturing, manufacturing, expanding, differentiating and/or reprogramming cells. For example, in suitable embodiments, the 3D hydrogel scaffold is prepared as a thermoreversible hydrogel scaffold using polymers such as for example poly(ethylene glycol)-(N-isopropylacrylamide) and the like. In yet other suitable embodiments, the 3D hydrogel scaffold is prepared from alginate polymers. Suitable alginate polymers include any commercially available or home-purified alginate polymer, such as alginate acid or sodium alginate from Sigma (+W201502), and modified alginate polymers, such as methacrylate modified alginate.

Generally, the 3D hydrogel scaffold for use in the closed housings of the devices of the present disclosure are in any form as known in the art, including, by way of example, sheets, fibers, hollow fibers, spheres, and combinations thereof.

Generally, cells are encapsulated in the hydrogel scaffold. In some suitable embodiments, cells are suspended in the hollow space created by the hydrogel hollow fibers. Cells include primary cells isolated from humans, such as T cells, chondrocytes, mesenchymal stem cells. Cells also include human induced pluripotent stem cells, human embryonic stem cells and their derivatives (i.e. cell differentiated from them). Cells also include primary human tumor cells. While described herein in the context of human cells, it should be understood by one skilled in the art that the device of the present disclosure can be used with any other animal cells without departing from the scope of the present disclosure.

Further, in one embodiment, the cells are autologous cells in that they are cells from the same patient desired to be treated. In another embodiment, the cells are allogenic cells (e.g., formed in another location and transported).

The device of the present disclosure further includes an inlet 120 and an outlet 130. The inlet 120 allows for entry of a cell culture medium into the closed housing 110, and the outlet 130 allows for exit of the cell culture medium from the closed housing 110. In particular embodiments, it is advantageous to include a pump (not shown) in flow communication with the inlet 120 to thereby pump cell culture medium from a medium reservoir 124 to the closed housing 110. While described in communication with a pump, it should be understood by one skilled in the art that any means of flowing the cell culture medium from medium reservoir 124 to the closed housing 110 can be used in the device 100 of the present disclosure without departing from the scope of the present disclosure.

Once used for cell culturing, the cell culture medium is automatically perfused through the closed housing 110 and exhausted from the closed housing 110 via the outlet 130 to an exhausted medium reservoir 132.

The cell culture medium can be any medium known in the cell culture art that is suitable for supporting cell survival, growth, expansion, and differentiation. Typically, the cell culture medium will include, but is not limited to, a carbon source, a nitrogen source, and growth factors. The specific cell culture medium for use in culturing the cells will depend on the cell type to be cultured. Cell culture conditions will also vary depending on the type of cell, the amount of cell expansion, and the number of cells desired.

### Methods of Culturing/Manufacturing/Expanding/Differentiating/Reprogramming Cells

The methods of the present disclosure may be used to culture cells on a personalized scale. As used herein, “culturing cells” or “culture cells” or the like refers to manufacturing, expanding, differentiating, and/or reprogramming cells within the device of the present disclosure. “Reprogramming” or “reprogram” refers to the conversion of adult cells back to iPSCs, or from one adult cell type to another cell type. The methods of the present disclosure provide at least the following advantages over conventional cell culture methods: (1) allow for biomanufacturing cells at high volumetric yield. At least 2×10⁷ cells can be produced per ml of hydrogel scaffold. In general, 5.0×10⁸ cells can be produced per ml of hydrogel scaffold; (2) allow for personalized medicine with miniature device at the point-of-care; (3) allow for limited contamination and/or cross-contamination as the closed culturing and point-of-care procedure removes the risk of contamination during cell culture transportation; and (4) allow for low batch-to-batch variation. Further, the methods of using the hydrogel scaffold for expanding and differentiating cells provide the additional benefits of: (1) providing 3D spaces for cell growth; and (2) providing physical barriers to prevent cell agglomeration and isolate shear force, major factors of which lead to low cell growth and volumetric yield of cells in the conventional 3D suspension culture technologies. The methods of using the device for reprogramming cells provide the additional benefit of allowing only the successfully reprogrammed cells to grow in the 3D hydrogel scaffold, thus generating cells at high purity.

Non-limiting examples of such cells that can be cultured, manufactured, expanded, differentiated, and/or reprogrammed using the methods and devices described herein include primary cells isolated from human (i.e., human primary cells) such as T cells, chondrocytes, and mesenchymal stem cells. Cells also include human induced pluripotent stem cells, human embryonic stem cells and their derivatives (i.e. cell differentiated from them). Cells also include primary human tumor cells. Cells can also be animal cells, for instance pig induced pluripotent stem cells or primary pig cells. While described more fully using iPSCs, it should be recognized that the methods and devices described herein can be used with any of the above-listed types of cells without departing from the scope of the present disclosure.

In general, the method of culturing cells includes: encapsulating cells in the hydrogel scaffolds or suspending cells in the hollow space created by the hydrogel hollow fibers of the closed housing; introducing a cell culture medium into the closed housing including the cells suspended in the hydrogel scaffolds to allow expansion, differentiation or reprogramming of the cells; and culturing the cells.

Cells are encapsulated or suspended in hydrogel scaffolds at concentrations varying from 1 to a few billion cells per milliliter and can be expanded to up to 6.0×10⁸ cells per milliliter.

In suitable embodiments, cells are encapsulated in the hydrogel scaffold. In other suitable embodiments, cells are suspended in the hollow space created by the hydrogel hollow fibers.

Cell culture medium is then introduced into the closed housing for culturing the cells. The cell culture medium can be any medium known in the cell culture art that is suitable for supporting cell survival, growth, expansion, differentiation and reprogramming Typically, the cell culture medium will include, but is not limited to, a carbon source, a nitrogen source, and growth factors. The specific cell culture medium for use in culturing the cells will depend on the cell type to be cultured.

Cell culture conditions will vary depending on the type of cell, the amount of cell expansion/differentiation/reprogramming, and the number of cells desired. Once sufficient cell expansion/differentiation/reprogramming and desired numbers of cells are reached, the cells are released from the 3D hydrogel scaffold by dissolving the 3D hydrogel scaffold chemically or physically within the housing. In one aspect, the scaffold is dissolved using a chemical dissolvent such as ethylenediaminetetraacetic acid (EDTA), ethylene glycol tetraacetic acid (EGTA), or an alginate lyase solution (available from Sigma-Aldrich). In another aspect, the hydrogel scaffold is dissolved using a physical method, such as lowering the temperature to below 4 ° C. The duration of the cells within the 3D hydrogel scaffold can typically vary from days to months.

The cells are useful in personalized medicine and can be used at the point-of-care. By way of example, the cells can be used in a procedure at the bedside of a patient. Cells can be efficiently and effectively prepared in size and number for use in degenerative disease and injury treatment, drug screening, for expressing proteins and the like. Additionally, the cells can be used to manufacture proteins and vaccines. In yet other aspects, the cells can be used for tissue engineering.

The disclosure will be more fully understood upon consideration of the following non-limiting Examples.

### EXAMPLES

Unless otherwise indicated, the hollow fibers were prepared as described above.

### Example 1

In this Example, expansion and growth of neural stem cells (NSCs) from induced pluripotent stem cells (iPSCs) were analyzed.

Methods

Miniature bioprocessing: With a syringe, 4° C. PNIPAAm-PEG solution containing iPSCs were injected into room temperature E8 medium in a 15-ml conical tube. Fibrous hydrogels were formed instantly. A Variable-Speed Peristaltic Tubing Pump (Control Company, USA) was used to continuously perfuse the culture medium into the tube through septum cap. Medium was stocked in a sealed and oxygen-permeable plastic bag. Medium in the bag was changed daily. The cell culture tube, pump and medium bag were placed in a cell culture incubator at 37° C. E8 medium and neural induction medium was used for days 1 to 5, and days 6 to 12, respectively. On day 12, the cell culture tube was placed on ice for 5 minutes to liquefy the hydrogel and release the spheroids. Cells were collected by spinning the tube at 100 g for 5 minutes. The cell pellet was treated with Accutase at 37° C. for 10 minutes and dissociated into single cells. Single cells were collected by spinning at 300 g for 5 minutes. Cells were resuspended with 80 μl PBS buffer and 20 μl of anti-SSEA-4 microbeads (Miltenyi Biotec) were added and incubated at 4° C. for 15 minutes. The SSEA4+ iPSCs were pulled down with a magnet and NSCs in the supernatant were transferred into a new tube. Cells were pelleted by spinning at 300 g for 5 minutes and transported to the surgery room for transplantation.

Cell transplantation: The animal experiments were carried out following the protocols approved by the University of Nebraska—Lincoln Animal Care and Use Committee. Sprague Dawley female rats were obtained from Charles River. Animals received intraperitoneal cyclosporine A (10 mg/kg, LC Laboratories) injection starting 1 day before transplantation. For transplantation, animals were anesthetized with 2-4% isoflurane. 2×10⁵ cells suspended in 4 μl DMEM medium were injected into striatum (AP+0.5 mm; ML±3.0 mm; DV-6 mm) at 0.5 μl/min using a 10 μl Hamilton syringe (Hamilton Company, USA) with a stereotaxic frame (RWD Life Science Inc). On day 7, rats were anesthetized with ketamine/xylazine and perfused with PBS followed by 4% paraformaldehyde. After fixation, the brain was serially sectioned (40 μm in thickness) with a Leica cryo-section machine, and free-floating sections were stained with antibodies.

To stain the brain sections, samples were then incubated with PBS +0.25% Triton X-100+5% goat serum+primary antibodies at 4° C. for 48 hours. After extensive wash, secondary antibodies in 2% BSA were added and incubated at 4° C. for 4 hours.

Results

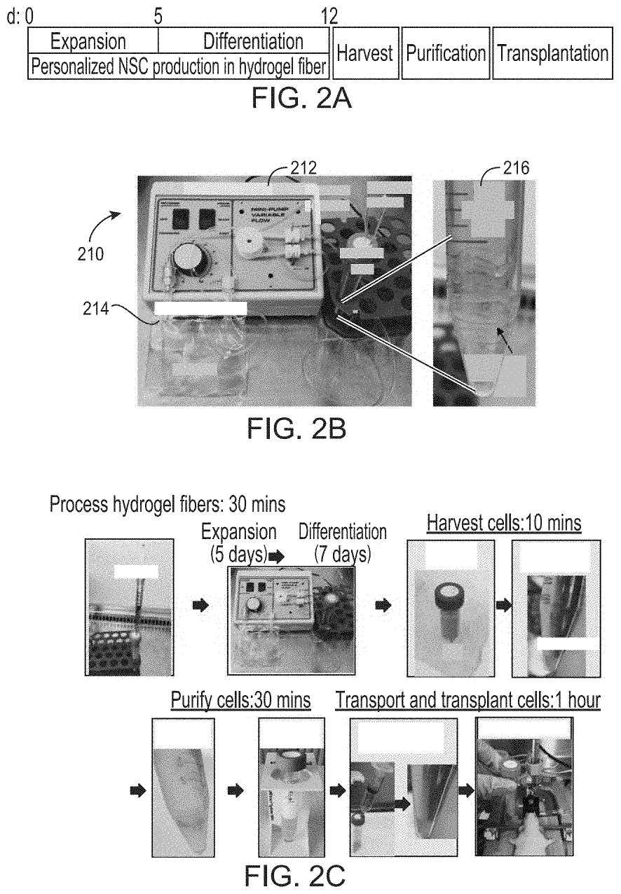

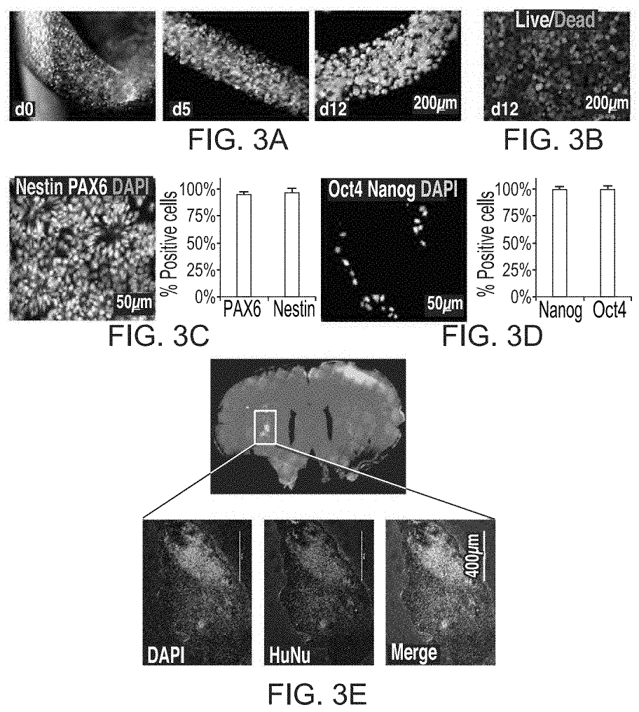

Taking advantage of the high cell yield in the PNIPAAm-PEG hydrogels, a prototype device of the present disclosure was built to make NSCs from hPSCs for personalized cell therapies (FIGS. 2A-2K and 3A-3E). On day 0, single iPSCs were mixed with 10% PNIPAAm-PEG solution at 4° C. With a syringe, the mixture was injected into room temperature E8 medium contained in a closed and sterile 15-ml conical tube with a septum cap (FIG. 2C). Fibrous hydrogels (with diameter ˜1 mm) were instantly formed with single iPSCs uniformly distributed in the hydrogels. The cells were cultured in a cell culture incubator at 37° C. and 5% CO₂. Medium stocked in a gas-permeable bag was continuously perfused into the cell culture tube (FIG. 2B). E8 medium was supplied for 5 days (FIG. 2C), followed by an additional 7 days of neural induction medium (FIG. 2C). On day 7, hydrogel scaffolds were liquefied by placing the cell culture tube on ice for 5 minutes (FIG. 2C). Cell spheroids were pelleted by spinning the tube at 100 g for 3 minutes (FIG. 2C). Medium was removed. Cell spheroids were incubated in Accutase at 37° C. for 10 minutes (FIG. 2C). Removing reagents from the tube and adding reagents to the tube were done with a sterile syringe through the septum. Magnetic beads coated with anti-SSEA4 antibodies were added into the tube to pull down the undifferentiated SSEA4+ iPSCs with a magnetic cell separator (FIG. 2C). Purified cells in the supernatant were transferred into a new, close tube (FIG. 2C) and transported to the surgical room. NSCs were transplanted to the brain of SCID mouse with a stereotactic injector (FIG. 2C).

Single iPSCs in hydrogel fibers grew into iPSC spheroids on day 5, and then became NSC spheroids on day 12 (FIG. 3A). With initial seeding density at 1×10⁶ cells/ml, 25-fold expansion and 2.5×10⁷ cells/ml hydrogel were achieved on day 7. A total of 1.0×10⁸ cells were produced in 4 ml of hydrogel in a 15-ml conical tube. Cell viability was >95% on day 7. 2% of the day 7 cells were SSEA4+. LIVE/DEAD® cell staining showed no or undetectable dead cells (FIG. 3B). After magnetic separation, the produced cells expressed PAX6 and Nestin (FIG. 3C) and Oct4+/Nanog+ cells were not detectable. Cells pulled down by the magnetic beads expressed both Oct4 and Nanog (FIG. 3D). 7 days after transplantation, large numbers of the human nuclear antigen positive (HuNu+) cells were found in the mouse brain (FIG. 3E).

### Example 2

In this Example, expansion and growth of neural stem cells (NSCs) from induced pluripotent stem cells (iPSCs) were analyzed.

Methods

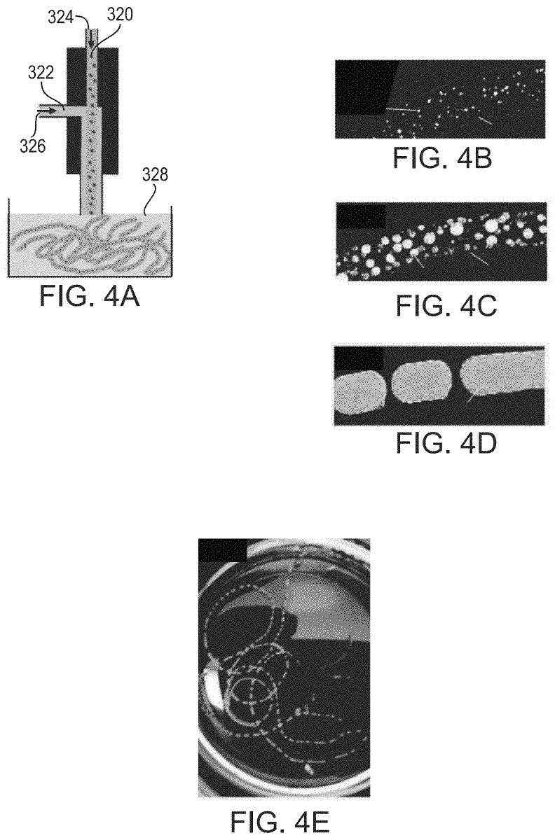

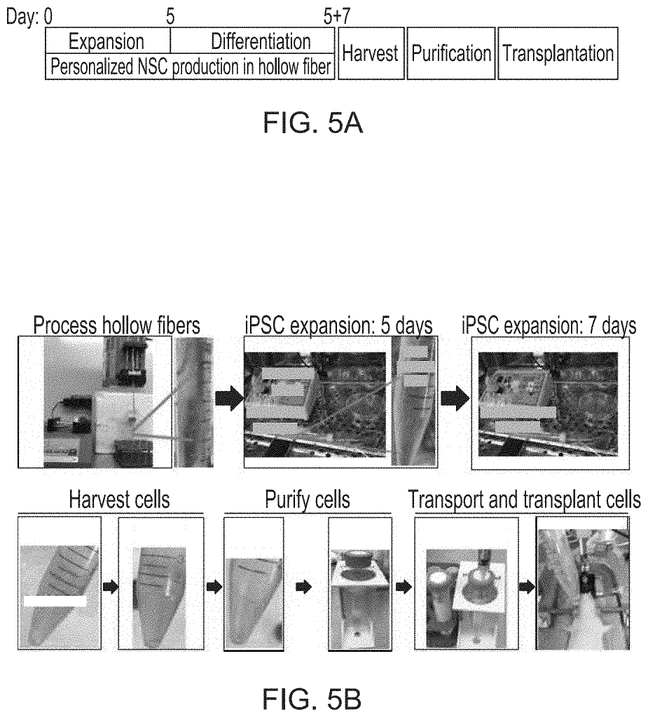

Miniature bioprocessing: a home-made micro-extruder was used to process alginate hollow fibers. A hyaluronic acid (HA) solution containing single cells and an alginate solution was pumped into the central and side channel of the home-made micro-extruder, respectively, and extruded into a CaCl₂ buffer (100 mM) in a closed 15-mL conical tube to make hollow fibers (FIGS. 4A, 5A & 5B). Subsequently, CaCl₂ buffer was replaced by cell culture medium. A Variable-Speed Peristaltic Tubing Pump (Control Company, USA) was used to continuously perfuse the culture medium into the tube through septum cap. Medium was stocked in a sealed and oxygen-permeable plastic bag. Medium in the bag was changed daily. The cell culture tube, pump and medium bag were placed in a cell culture incubator at 37° C. E8 medium and neural induction medium was used for days 1 to 5, and days 6 to 12, respectively. On day 12, 0.5 mM EDTA was pumped into the tube. The alginate hollow fibers were dissolved within 5 minutes. Cells were collected by spinning the tube at 100 g for 5 minutes. The cell pellet was treated with Accutase at 37° C. for 10 minutes and dissociated into single cells. Single cells were collected by spinning at 300 g for 5 minutes. Cells were resuspended with 80 μl PBS buffer and 20 μl of anti-SSEA-4 microbeads (Miltenyi Biotec) were added and incubated at 4° C. for 15 minutes. The SSEA4+ iPSCs were pulled down with a magnet and NSCs in the supernatant were transferred into a new tube. Cells were pelleted by spinning at 300 g for 5 minutes and transported to the surgery room for transplantation.

Cell transplantation: The animal experiments were carried out following the protocols approved by the University of Nebraska—Lincoln Animal Care and Use Committee. Sprague Dawley female rats were obtained from Charles River. Animals received intraperitoneal cyclosporine A (10 mg/kg, LC Laboratories) injection starting 1 day before transplantation. For transplantation, animals were anesthetized with 2-4% isoflurane. 2×10⁵ cells suspended in 4 μl DMEM medium were injected into striatum (AP+0.5 mm; ML±3.0 mm; DV-6 mm) at 0.5 μl/min using a 10 μl Hamilton syringe (Hamilton Company, USA) with a stereotaxic frame (RWD Life Science Inc). On day 7, rats were anesthetized with ketamine/xylazine and perfused with PBS followed by 4% paraformaldehyde. After fixation, the brain was serially sectioned (40 μm in thickness) with a Leica cryo-section machine, and free-floating sections were stained with antibodies.

To stain the brain sections, samples were then incubated with PBS +0.25% Triton X-100+5% goat serum+primary antibodies at 4° C. for 48 hours. After extensive wash, secondary antibodies in 2% BSA were added and incubated at 4° C. for 4 hours.

Results

Taking advantage of the high cell yield in the alginate hollow fibers, a prototype device of the present disclosure was built to make NSCs from hPSCs for personalized cell therapies (FIGS. 4A-4E, 5A & 5B). On day 0, single iPSCs were mixed with 1% HA solution. With a micro-extruder, the HA solution containing single cells 320 and an alginate solution 322 were pumped into the central 324 and side channel 326 of the micro-extruder, respectively, and extruded into a CaCl₂ buffer 328 (100 mM) in a closed 15-mL conical tube to make hollow fibers (FIG. 5B). The cells were cultured in a cell culture incubator at 37° C. and 5% CO₂. Medium stocked in a gas-permeable bag was continuously perfused into the cell culture tube (FIG. 5B). E8 medium was supplied for 5 days (FIG. 5B), followed by an additional 7 days of neural induction medium (FIG. 5B). On day 7, hydrogel scaffolds were liquefied by placing the cell culture tube on ice for 5 minutes (FIG. 5B). Cell spheroids were pelleted by spinning the tube at 100 g for 3 minutes (FIG. 5B). Medium was removed. Cell spheroids were incubated in Accutase at 37° C. for 10 minutes (FIG. 5B). Removing reagents from the tube and adding reagents to the tube were done with a sterile syringe through the septum. Magnetic beads coated with anti-SSEA4 antibodies were added into the tube to pull down the undifferentiated SSEA4+ iPSCs with a magnetic cell separator (FIG. 5B). Purified cells in the supernatant were transferred into a new, close tube (FIG. 5B) and transported to the surgical room. NSCs were transplanted to the brain of SCID mouse with a stereotactic injector (FIG. 5B).

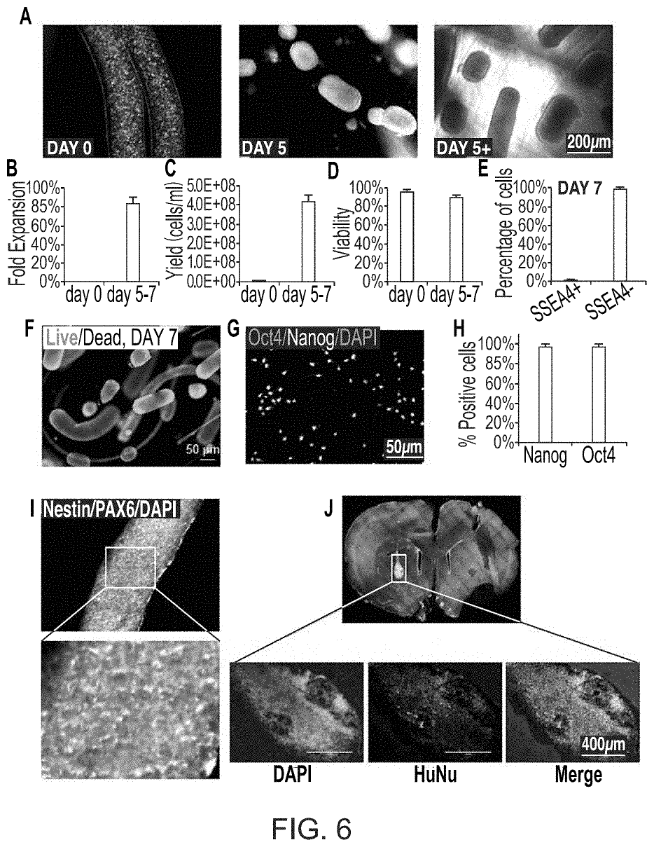

Single iPSCs in hydrogel fibers grew into iPSC spheroids on day 5, and then became NSC spheroids on day 12 (FIG. 6A). With initial seeding density at 1×10⁶ cells/ml, 400-fold expansion and 4.0×10⁸ cells/ml hydrogel were achieved on day 7. A total of 1.6×10⁹ cells were produced in 4 ml of hydrogel in a 15-ml conical tube. Cell viability was >95% on day 7. 2% of the day 7 cells were SSEA4+. LIVE/DEAD® cell staining showed no or undetectable dead cells (FIG. 6F). After magnetic separation, the produced cells expressed PAX6 and Nestin and Oct4+/Nanog+cells were not detectable. Cells pulled down by the magnetic beads expressed both Oct4 and Nanog (FIG. 6G). 7 days after transplantation, large numbers of the human nuclear antigen positive (HuNu+) cells were found in the mouse brain (FIG. 6J).

### Example 3

In this Example, human skin fibroblasts were reprogrammed into iPSCs using the methods and devices of the present disclosure.

Fibroblasts transfected with Episomal reprogramming vectors (e.g. EpiS™ Episomal iPSC Reprogramming Kit, ThemoFisher, Catalog number: A15960) were encapsulated and cultured in 3D thermoreversible PNIPAAm-PEG hydrogels prepared as described in Example 1 in E8 medium.

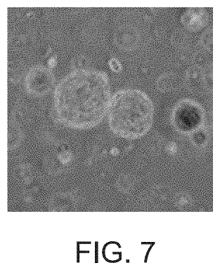

As shown in FIG. 7, pure iPSCs were produced within approximately 3 weeks.

These results demonstrated that the methods and devices of the present disclosure can be used to culture and manufacture cells. It is contemplated that the methods may be useful in both research laboratories, industries, and at the point-of-care for preparing sufficient and high quality cells for disease and injury treatments, screening libraries for drugs, and manufacturing proteins and vaccines.

1. A device for culturing cells for personalized medicine, the device comprising:
a closed housing comprising a three-dimensional (3D) hydrogel scaffold;
an inlet for introducing a cell culture medium into the housing; and
an outlet for exhausting cell culture medium from the housing.

2. The device as set forth at claim 1 wherein the closed housing is a closed cell culture tube.

3. The device as set forth in claim 1 wherein the closed housing has a capacity of less than 10 L.

4. The device as set forth in claim 1 wherein the 3D hydrogel scaffold comprises at least one of poly(ethylene glycol) (PEG)-(N-isopropylacrylamide) polymers and alginate polymers.

5. (canceled)

6. The device as set forth in claim 1 wherein the 3D hydrogel scaffold is in a form selected from the group consisting of a sheet, fiber, hollow fiber, sphere, and combinations thereof.

7. The device as set forth in claim 1 wherein the cells are selected from the group consisting of human primary cells, induced pluripotent stem cells (iPSCs), embryonic stem cells, and derivatives thereof, primary tumor cells, and combinations thereof.

8. (canceled)

9. A method of expanding cells using the device as set forth in claim 1, the method comprising:
suspending a cell solution including cells in the 3D hydrogel scaffold of the closed housing;
introducing a cell culture medium into the closed housing from the inlet; and
culturing the cells.

10. The method as set forth in claim 9 wherein suspending the cell solution including cells comprises encapsulating cells from the cell solution into the 3D hydrogel scaffold.

11. The method as set forth in claim 9 further comprising releasing the cultured cells from the 3D hydrogel scaffold comprising dissolving the 3D hydrogel scaffold, wherein the 3D hydrogel scaffold is dissolved by at least one of: using a chemical dissolvent selected from the group consisting of ethylenediaminetetraacetic acid (EDTA), ethylene glycol tetraacetic acid (EGTA), and an alginate lyase solution and using mechanical force.

12. (canceled)

13. (canceled)

14. The method as set forth in claim 9 further comprising purifying the cultured cells by contacting the cultured cells with antibody-coated magnetic beads within the closed housing.

15. The method as set forth in claim 9 further comprising concentrating the cultured cells by centrifuging the cultured cells or contacting the cultured cells with antibody-coated magnetic beads within the closed housing.

16. (canceled)

17. The device as set forth in claim 9 wherein the 3D hydrogel scaffold comprises at least one of poly(ethylene glycol) (PEG)-(N-isopropylacrylamide) polymers and alginate polymers.

18. (canceled)

19. (cancelled)

20. A method of differentiating cells using the device as set forth in claim 1, the method comprising:
suspending a cell solution including cells in the 3D hydrogel scaffold of the closed housing;
introducing a cell differentiation medium into the closed housing from the inlet; and
culturing the cells.

21. The method as set forth in claim 20 wherein suspending the cell solution including cells comprises encapsulating cells from the cell solution into the 3D hydrogel scaffold.

22. The method as set forth in claim 20 further comprising releasing the cultured cells from the 3D hydrogel scaffold comprising dissolving the 3D hydrogel scaffold, wherein the 3D hydrogel scaffold is dissolved by at least one of: using a chemical dissolvent selected from the group consisting of ethylenediaminetetraacetic acid (EDTA), ethylene glycol tetraacetic acid (EGTA), and an alginate lyase solution and using mechanical force.

23. (canceled)

24. (canceled)

25. The method as set forth in claim 20 further comprising purifying the cultured cells by contacting the cultured cells with antibody-coated magnetic beads within the closed housing.

26. The method as set forth in claim 20 further comprising concentrating the cultured cells by centrifuging the cultured cells or contacting the cultured cells with antibody-coated magnetic beads within the closed housing.

27. (canceled)

28. The device as set forth in claim 20 wherein the 3D hydrogel scaffold comprises at least one of poly(ethylene glycol) (PEG)-(N-isopropylacrylamide) polymers and alginate polymers.

29. (canceled)

30. (canceled)

31. A method of reprogramming cells using the device as set forth in claim 1, the method comprising:
suspending a cell solution including adult cells in the 3D hydrogel scaffold of the closed housing;
introducing a cell culture medium into the closed housing from the inlet; and
reprogramming the cells.

32. The method as set forth in claim 31 wherein suspending the cell solution including cells comprises encapsulating cells from the cell solution into the 3D hydrogel scaffold.

33. The method as set forth in claim 31 further comprising releasing the cultured cells from the 3D hydrogel scaffold comprising dissolving the 3D hydrogel scaffold, wherein the 3D hydrogel scaffold is dissolved by at least one of: using a chemical dissolvent selected from the group consisting of ethylenediaminetetraacetic acid (EDTA), ethylene glycol tetraacetic acid (EGTA), and an alginate lyase solution and using mechanical force.

34. (canceled)

35. (cancelled)

36. The method as set forth in claim 31 further comprising purifying the cultured cells by contacting the cultured cells with antibody-coated magnetic beads within the closed housing.

37. The method as set forth in claim 31 further comprising concentrating the cultured cells by centrifuging the cultured cells or contacting the cultured cells with antibody-coated magnetic beads within the closed housing.

38. (canceled)

39. The device as set forth in claim 31 wherein the 3D hydrogel scaffold comprises at least one of poly(ethylene glycol) (PEG)-(N-isopropylacrylamide) polymers and alginate polymers.

40. (canceled)

41. (canceled)
